(19) United States
(12) Patent Application Publication
(10) Pub. No.: US 2023/0380771 A1
(43) Pub. Date: Nov. 30, 2023

(54) CLASSIFYING TIME SERIES USING RECONSTRUCTION ERRORS

(71) Applicant: X Development LLC, Mountain View, CA (US)

(72) Inventors: Sarah Ann Laszlo, Mountain View, CA (US); David Passey, Chapel Hill, NC (US)

(21) Appl. No.: 17/825,427

(22) Filed: May 26, 2022

(51) Int. Cl. A61B 5/00

(52) U.S. Cl. CPC A61B 5/7267; A61B 5/7275; A61B 5/7285; A61B 5/725

(57) ABSTRACT

Methods, systems, and apparatus, including computer programs encoded on a computer storage medium, for classifying an input time series into a class from a set of classes. In one aspect, a method comprises: receiving an input time series; processing the input time series using a reconstruction model to generate a reconstruction model output that comprises a plurality of channels, wherein each channel of the plurality of channels defines a respective output time series, and wherein each channel of the plurality of channels corresponds to a respective class from the set of classes; determining a respective reconstruction error for each channel of the plurality of channels based on an error between: (i) the output time series defined by the channel, and (ii) the input time series; and classifying the input time series as being included in a class from the set of classes based on the reconstruction errors.

### BACKGROUND

This specification relates to processing data using machine learning models.

Machine learning models receive an input and generate an output, e.g., a predicted output, based on the received input. Some machine learning models are parametric models and generate the output based on the received input and on values of the parameters of the model.

Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes a classification system implemented as computer programs on one or more computers in one or more locations that can classify an input time series as being included in a class from a set of classes.

More specifically, the classification system can process the input time series using a reconstruction model to generate a reconstruction model output that includes a set of channels. Each channel corresponds to a respective class from the set of classes and defines a respective output time series that is a predicted reconstruction of the input time series. The classification system determines a respective reconstruction error for each channel of the reconstruction model output based on an error between: (i) the output time series defined by the channel, and (ii) the input time series. The classification system then classifies the input time series as being included in a class from the set of classes based on the reconstruction errors. For example, the classification system can classify the input time series as being included in a class corresponding to a channel with the lowest reconstruction error.

According to a first aspect, there is provided a method performed by one or more computers for classifying an input time series into a class from a set of classes, the method comprising: receiving an input time series comprising a respective sample at each time point in a sequence of time points; processing the input time series using a reconstruction model to generate a reconstruction model output that comprises a plurality of channels, wherein each channel of the plurality of channels defines a respective output time series that is a predicted reconstruction of the input time series, and wherein each channel of the plurality of channels corresponds to a respective class from the set of classes; determining a respective reconstruction error for each channel of the plurality of channels based on an error between: (i) the output time series defined by the channel, and (ii) the input time series; and classifying the input time series as being included in a class from the set of classes based on the reconstruction errors.

In some implementations, classifying the input time series as being included in a class from the set of classes based on the reconstruction errors comprises: identifying a class corresponding to a channel with a lowest reconstruction error from among the plurality of channels; and classifying the input time series as being included in the identified class.

In some implementations, the reconstruction model comprises: (i) a transformation model including a set of transformation functions, and (ii) a projection model, and processing the input time series using the reconstruction model to generate the reconstruction model output comprises: processing the input time series using the transformation model to generate a collection of transformed time series, wherein each transformed time series results from applying a respective transformation function from the set of transformation functions to the input time series; and processing the collection of transformed time series using the projection model to generate the reconstruction model output.

In some implementations, the set of transformation functions comprises one or more non-linear transformation functions.

In some implementations, the set of transformation functions comprises one or more of: a high-pass filter transformation function, a low-pass filter transformation function, a band-pass filter transformation function, a constant transformation function, an identity transformation function, or a lagging transformation function.

In some implementations, processing the collection of transformed time series using the projection model to generate the reconstruction model output comprises: generating each channel of the reconstruction model output as a respective linear combination of the collection of transformed time series.

In some implementations, each transformed time series comprises a same number of samples as the input time series.

In some implementations, the reconstruction model has been trained on a set of training time series, and the training encourages that, for each training time series, a channel of a reconstruction model output for the training time series that corresponds to a class of the training time series has a lower reconstruction error than each other channel of the reconstruction model output for the training time series.

In some implementations, the training comprises, for each training time series: generating a target output for the training time series, wherein the target output comprises a respective channel corresponding to each class from the set of classes, wherein: the channel of the target output corresponding to a class of the training time series defines the training time series; and each channel of the target output corresponding to a class different from the class of the training time series defines a default time series; and training the reconstruction model to minimize an error between: (i) a reconstruction model output generated by processing the training time series using the reconstruction model, and (ii) the target output for the training time series.

In some implementations, the default time series has a constant value of zero.

In some implementations, the transformation model comprises a set of transformation model parameters, the projection model comprises a set of projection model parameters, and training the reconstruction model comprises: training the projection model parameters while maintaining the transformation model parameters as static values.

In some implementations, for each channel of the plurality of channels, the reconstruction error is based on an L₂ error between: (i) the output time series defined by the channel, and (ii) the input time series.

In some implementations, the method further comprises determining that the classification of the input time series satisfies a level of confidence defined by an error threshold.

In some implementations, determining that the classification of the input time series satisfies the level of confidence defined by the error threshold comprises: determining that a reconstruction error for the channel corresponding to the class into which the input time series has been classified is below the error threshold.

In some implementations, the input time series represents an audio waveform.

In some implementations, the input time series represents radar data.

In some implementations, the input time series represents a biomedical signal.

In some implementations, the biomedical signal comprises one or more of: a blood pressure signal, an electroencephalography (EEG) signal, an electrocardiogram (ECG) signal, or an electromyography (EMG) signal.

According to another aspect, there is provided a system comprising: one or more computers; and one or more storage devices communicatively coupled to the one or more computers, wherein the one or more storage devices store instructions that, when executed by the one or more computers, cause the one or more computers to perform operations of the methods described herein.

One or more non-transitory computer storage media storing instructions that when executed by one or more computers cause the one or more computers to perform operations described herein.

Particular embodiments of the subject matter described in this specification can be implemented so as to realize one or more of the following advantages.

The classification system can classify input time series more rapidly and using significantly fewer parameters than conventional systems achieving comparable accuracy. For example, the classification system can implement a reconstruction model that includes: (i) a transformation model that generates a collection of transformed versions of the input time series, and (ii) a projection model that generates each output time series as a linear combination of the transformed time series. The transformation model does not require training, while the projection model can be implemented as a single matrix trained by an efficient one-step optimization. In contrast, conventional systems can include large numbers of parameters that require training over many time steps using iterative optimization procedures, e.g., stochastic gradient descent. The lightweight design of the classification system enables accurate classification of times series while requiring minimal power resources, thus making the classification system suitable for deployment in low-power and resource-constrained environments, such as on mobile devices and implanted medical devices.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example classification system.

FIG. 2 shows an example reconstruction model.

FIG. 3 provides an illustration of example operations that can be performed by the classification system.

FIG. 4 is a flow diagram of an example process for classifying an input time series into a class from a set of classes.

FIG. 5 is a flow diagram of an example process training a reconstruction model.

Like reference numbers and designations in the various drawings indicate like elements.

### DETAILED DESCRIPTION

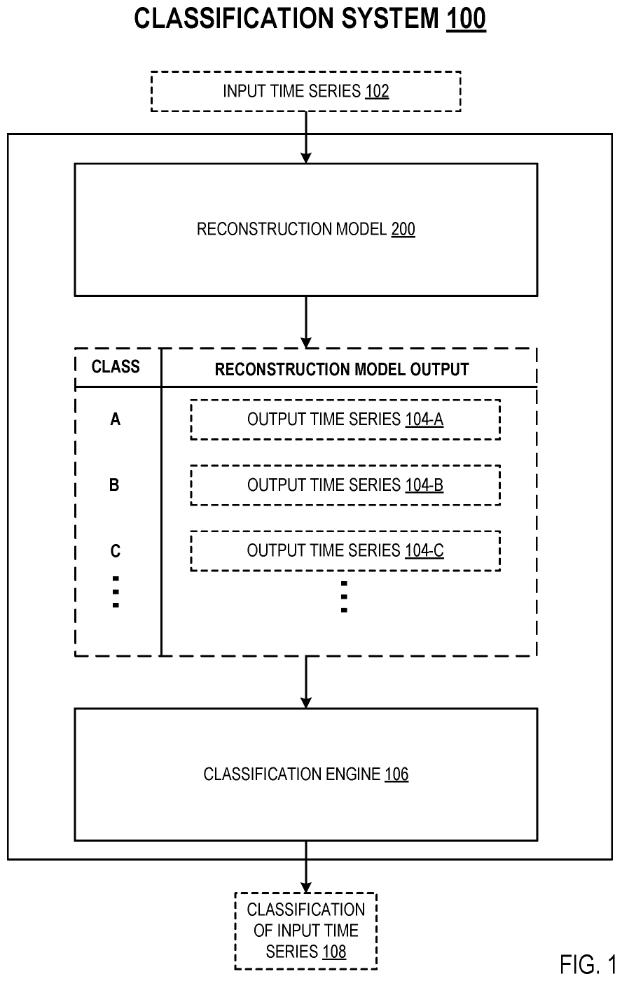

FIG. 1 shows an example classification system 100. The classification system 100 is an example of a system implemented as computer programs on one or more computers in one or more locations in which the systems, components, and techniques described below are implemented.

The classification system 100 is configured to process an input time series 102 to generate a classification 108 of the input time series 102. The classification 108 of the input time series 102 designates the input time series 102 as being included in a class from a set of classes.

The input time series 102 includes a respective sample at each time point in a sequence of time points. Each sample can be represented as an ordered collection of one or more numerical values, e.g., as a scalar, a vector, or a matrix of numerical values. The sequence of time points can include any appropriate number of time points, e.g., 1000 time points, 10,000 time points, or 100,000 time points.

The input time series 102 can represent any appropriate type of signal. A few examples of input time series 102 are described next.

In some implementations, the input time series 102 can represent an audio waveform, e.g., captured using a microphone, and each sample in the input time series 102 can represent an audio sample at a respective time point.

In some implementations, the input time series 102 can represent radar data, e.g., generated by a radar or radar array, and each sample in the input time series 102 can represent radar measurements captured at a respective time point.

In some implementations, the input time series 102 can represent a biomedical signal that characterizes physiological activity in the body of a subject.

For example, the input time series 102 can represent a blood pressure signal, e.g., captured using a blood pressure monitor, and each sample in the input time series 102 can represent systolic or diastolic blood pressure in a subject at a respective time point.

In another example, the input time series 102 can represent an electroencephalography (EEG) signal, e.g., measured using one or more probes placed on the scalp of a subject, that characterizes neural activity in the brain of subject. In this example, each sample in the input time series 102 can represent electrical activity (e.g., voltage) measurements obtained by the probes at a respective time point.

In another example, the input time series 102 can represent an electrocardiogram (ECG) signal, e.g., measured using one or more probes placed on the skin of a subject, that characterizes electrical activity in the heart of the subject. In this example, each sample in the input time series 102 can represent electrical activity (e.g., voltage) measurements obtained by the probes at a respective time point.

In another example, the input time series 102 can represent an electromyography (EMG) signal, e.g., captured using a needle electrode placed in a muscle of a subject, that characterizes electrical activity in the muscle. In this example, each sample in the input time series 102 can represent electrical activity (e.g., voltage) measurement obtained by the needle electrode at a respective time point.

In some implementations, the input time series 102 can represent video data, in particular, a sequence of video frames of a video, e.g., captured using a video camera. Each sample in the input time series 102 can represent a video frame at a respective time point, e.g., as a vector generated by concatenating pixels representing the video frame in a defined order.

The input time series 102 can be captured by one or more sensors located in any appropriate location, e.g., on a user device, e.g., a smartwatch, smartphone, personal digital assistant, or the like, or on a medical device, e.g., a blood pressure monitor, an EEG machine, an ECG machine, or an EMG machine. In some instances, the input time series can be captured by sensors located in a medical device implanted in the body of subject, e.g., in the brain of the subject, or in the heart of the subject.

The classification system 100 can classify the input time series 102 into any appropriate set of classes. A few examples of classes are described next.

In some implementations, the input time series 102 can represent an audio waveform, and the set of classes can include a respective class corresponding to each of one or more classes of sounds. For example, the set of classes can include a class corresponding to a wake-word for a personal digital assistant. As another example, the set of classes can include respective classes corresponding to one or more commands, e.g., “stop,” “start,” “fast forward,” “rewind,” etc. As another example, the set of classes can include a class corresponding to one or more types of sound, e.g., crying baby, dog barking, siren, etc. In these implementations, the audio waveform can be classified as being included in a class corresponding to a class of sound if the audio waveform represents a sound included in the class of sound.

In some implementations, the input time series 102 can represent radar data or video data, and the set of classes can include a respective class corresponding to each of one or more types of gestures, e.g., “swipe up,” “swipe down,” “swipe left,” “swipe right,” etc. In this implementation, the radar data or video data can be included in a class corresponding to a gesture if the radar data or video data characterizes a motion that performs the gesture.

In some implementations, the input time series 102 can represent a biomedical signal characterizing a subject, and the set of classes can include a respective class corresponding to each of one or more medical conditions. In these implementations, the input time series 102 can be included in a class corresponding to a medical condition if the subject has the medical condition. For example, the input time series 102 can represent an EEG signal characterizing the brain of a subject, and the set of classes can include classes corresponding to one or more of: epilepsy, concussion, sleep apnea, or dementia. As another example, the input time series 102 can represent an ECG signal characterizing the heart of a subject, and the set of classes can include classes corresponding to one or more of: fibrillation, tachycardia, or bradycardia. As another example, the input time series 102 can represent an EMG signal characterizing a muscle of a subject, and the set of classes can include classes corresponding to one or more of: one or more muscle disorders (e.g., inflammatory myopathy), one or more nerve disorders (e.g., carpal tunnel syndrome), or one or more plexus disorders (e.g., neuralgic amyotrophy).

The set of classes can include any appropriate number of classes, e.g., 2 classes, 10 classes, or 100 classes. Optionally, the set of classes can include a “default” class, where an input time series is designated as belonging to the default class if the input time series does not belong to any of the other classes in the set of classes. For instance, if the set of classes include classes corresponding to medical conditions, then the default class can represent a “healthy” class. In this instance, an input time series 102 representing a biomedical signal characterizing a subject can be included in the “healthy” class if the subject does not have medical conditions corresponding to the other classes in the set of classes.

The classification system 100 can process an input time series 102 to generate a classification 108 of the input time series 102 using a reconstruction model 200 and a classification engine 106, which are each described in more detail next.

The reconstruction model 200 is configured to process an input time series 102, in accordance with values of a set of reconstruction model parameters, to generate a reconstruction model output that includes a respective channel corresponding to each class in the set of classes. (A “channel” can be represented as an ordered collection of numerical values, e.g., a vector, matrix, or other tensor of numerical values). Each channel in the reconstruction model output defines a respective output time series that is a predicted reconstruction (e.g., estimate) of the input time series 102. In the example illustrated in FIG. 1, the reconstruction model output can include an output time series 104-A corresponding to “class A,” an output time series 104-B corresponding to “class B,” an output time series 104-C corresponding to “class C,” and so on.

The classification system 100 can train the reconstruction model 200 to encourage the channel of the reconstruction model output that corresponds to the class of the input time series 102 to have a lower reconstruction error than the other channels of the reconstruction model output. That is, the classification system 100 can train the reconstruction model 200 to encourage the channel of the reconstruction model output that corresponds to the class of the input time series 102 to reconstruct the input time series 102 more accurately than the other channels of the reconstruction model output.

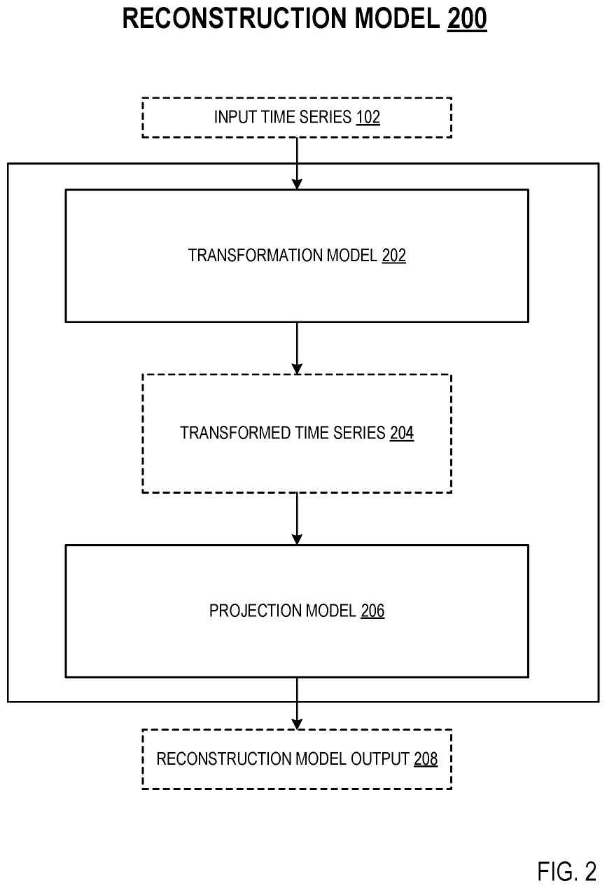

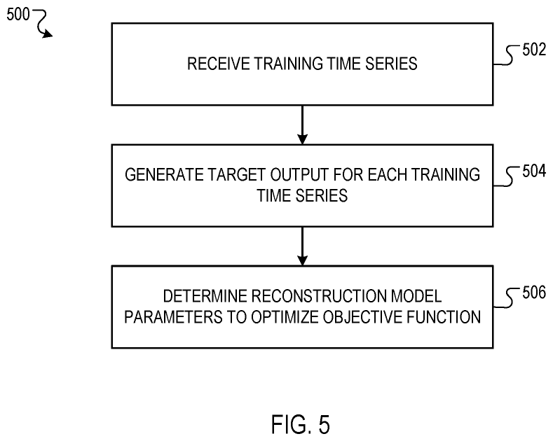

An example architecture of the reconstruction model is described in more detail below with reference to FIG. 2. An example technique for training the reconstruction model is described in more detail with reference to FIG. 5.

The classification engine 106 is configured to process the reconstruction model output to generate a classification 108 of the input time series 102 into a class from the set of classes.

To generate the classification 108 of the input time series 102, the classification engine 106 can process the reconstruction model output to generate a respective reconstruction error for each channel of the reconstruction model output. A reconstruction error for a channel of the reconstruction model output measures an error between: (i) the output time series defined by the reconstruction model output, and (ii) the input time series 102, e.g., using an L₁ error, an L₂ error, or any other appropriate measure of error. For instance, the classification engine 106 can generate the reconstruction error Eᵢ for channel i of the reconstruction model output as:

Eᵢ=∥I−Oᵢ∥₂ (1)

where I denotes the input time series, Oᵢ denotes the output time series defined by channel i of the reconstruction model output, and ∥⋅∥₂ denotes an L₂ norm. Generally, a reconstruction error for a channel of the reconstruction model can be represented as a scalar numerical value.

The classification engine 106 can determine the classification 108 of the input time series 102 based on the reconstruction errors for the channels of the reconstruction model output. For example, the classification engine 106 can identify the input time series 102 as being included in the class corresponding to the channel of the reconstruction model output having the lowest reconstruction error. Thus the classification engine 106 can classify the input time series 102 as being included in the class corresponding to the channel of the reconstruction model output that most accurately reconstructs the input time series 102.

In some implementations, as part of classifying the input time series 102, the classification engine 106 can compare the reconstruction error of the channel of the reconstruction model output that most accurately reconstructs the input time series to a predefined error threshold. If the classification engine 106 determines that the reconstruction error of the channel that most accurately reconstructs the input time series exceeds the error threshold, then the classification engine 106 can refrain from classifying the input time series 102. Rather, the classification engine 106 can output a notification indicating that the input time series 102 cannot be classified into a class from the set of classes with a level of confidence defined by the error threshold. The error threshold can be specified in any appropriate manner, e.g., by a user of the classification system 100.

The classification system 100 can be used in any of a variety of applications. A few example applications of the classification system 100 are described next.

In some implementations, the classification system 100 can process biomedical signals generated by a device, e.g., a device worn by a subject (e.g., a smartwatch), or a device implanted in a subject (e.g., in the brain or the heart of the subject). For instance, the classification system 100 can process electrical signals obtained by a sensor implanted in the brain of a subject to classify whether the subject is likely to experience a seizure, e.g., within a predefined window of time, e.g., 5 minutes, 30 minutes, or 60 minutes. As another example, the classification system 100 can process electrical signals obtained by a sensor implanted in the heart of a subject to classify whether the subject is likely to experience a cardiac event (e.g., heart attack), e.g., within a predefined window of time, e.g., 5 minutes, 30 minutes, or 60 minutes. If the classification system 100 generates a classification 108 indicating that the subject may require medical attention, the device can notify the subject, or automatically transmit a request for medical assistance (e.g., to an emergency response service), or both.

In some implementations, the classification system 100 can process signals (e.g., representing audio data, radar data, or video data) generated by sensors of a personal digital assistant. For instance, the classification system 100 can process an audio signal generated by sensors of a personal digital assistant to classify whether a user has spoken a predefined “wake-word.” If the classification system 100 generates a classification 108 indicating the user has spoken a wake-word, then the personal digital assistant can activate, e.g., by monitoring an audio sensor to determine if the user issues one or more commands, e.g., to send a text or to set a timer. As another example, the classification system 100 can process a radar signal or a video signal generated by sensors of a personal digital assistant to classify whether a user has made a predefined gesture. If the classification system 100 generates a classification 108 indicating that the user has made a gesture, the personal digital assistant can take one or more actions in response to having detected the gesture. For instance, in response to detecting a “swipe right” gesture, the personal digital assistant can advance to a next song in playlist, or can advance to a next page in an e-book being displayed to the user.

FIG. 2 shows an example reconstruction model 200, e.g., that is included in the classification system 100 described with reference to FIG. 1. The reconstruction model 200 is configured to process an input time series 102 to generate a reconstruction model output that includes a respective channel corresponding to each class in the set of classes. Each channel in the reconstruction model output defines a respective output time series that is a predicted reconstruction of the input time series 102.

The reconstruction model 200 includes a transformation model 202 and a projection model 206, which are each described in more detail next.

The transformation model 202 is configured to process the input time series 102 to generate a set of transformed time series 204. More specifically, the transformation model 202 can generate each transformed time series 204 by applying a respective transformation function to the input time series 102. Each transformed time series 204 can have the same number of samples as the input time series 102.

The transformation model 202 can be configured to generate any appropriate number of transformed times series 204, e.g., 10, 100, or 1000 transformed time series 204. The transformation model 202 can generate the transformed time series 204 using any appropriate transformation functions. A few examples of transformation functions are described next.

In some implementations, the transformation model 202 can implement an “constant” transformation function. The constant transformation function can map the input time series 102 to a predefined default time series, i.e., that does not depend on the input time series, e.g., a time series having a constant value of “1” in every entry of a tensor defining the time series.

In some implementations, the transformation model 202 can implement an “identity” transformation function. The identity transformation function can map the input time series 102 to an identical time series, i.e., such that the set of transformed time series 204 includes the input time series 102 itself.

In some implementations, the transformation model 202 can implement a “filtering” transformation function, e.g., that generates a transformed time series by applying a filtering operation to the input time series 102. More specifically, the transformation function can apply a filtering operation to the input time series 102 by convolving a filtering kernel (e.g., represented as a tensor of numerical values) with the input time series. For example, the filtering operation can be a “low-pass” filtering operation that attenuates high-frequency components of the input time series, e.g., a 10^{th} order low-pass Butterworth filter with a cutoff at 1000 Hz. As another example, the filtering operation can be a “high-pass” filtering operation that attenuates low-frequency components of the input time series, e.g., a 10^{th} order high-pass Butterworth filter with a cutoff at 1000 Hz. As another example, the filtering operation can be a “band-pass” filtering operation that attenuates frequency components of the input time series that fall outside a defined range of frequencies.

In some implementations, the transformation model 202 can implement an element-wise non-linear transformation function, e.g., that operates independently on each element of a tensor defining the input time series 102. For example, the transformation function can apply an arctan function or a sigmoid function separately to each element of the input time series 102.

In some implementations, the transformation model 202 can implement a “lagging” transformation function, e.g., that deletes one or more samples from one end of the input time series 102, and adds an equivalent number of default (i.e., predefined) samples to the other end of the input time series 102.

In some implementations, the transformation model 202 can implement a “composed” transformation function that is defined as a composition of multiple constituent transformation functions. For example, the transformation model 202 can implement a composed transformation function that is defined as a composition of a lagging transformation function with a filtering transformation function.

In some implementations, the transformation model 202 can implement a “random” transformation function that is parametrized by a set of randomly chosen parameters, e.g., parameters that are sampled in accordance with a probability distribution, e.g., a standard Normal distribution. For example, the transformation model can implement a random transformation that is parameterized by a random matrix (i.e., a matrix composed of randomly sampled elements), where the random transformation operates on the input time series by matrix multiplying the input time series by the random matrix. Optionally, the random transformation function can apply an element-wise non-linear transformation (e.g., an arctan transformation or a sigmoid transformation) to the time series resulting from matrix multiplying the input time series by the random matrix.

The projection model 206 is configured to process the set of transformed time series 204 to generate the reconstruction model output. In particular, the projection model 206 can generate each channel of the reconstruction model output as a respective combination of the set of transformed time series. For example, the projection model 206 can generate each channel of the reconstruction model output 208 as a linear combination of the time series in the set of transformed time series 204.

The reconstruction model 200 is parametrized by a set of reconstruction model parameters, including: (i) the parameters of the transformation model 202, and (ii) the parameters of the projection model 206. The classification system 100 can train the reconstruction model parameters to encourage that, for any input time series, the channel of the reconstruction model output that corresponds to the class of the input time series has a lower reconstruction error than the other channels of the reconstruction model output.

Optionally, the classification system 100 can train the parameters of the projection model 206, while maintaining the parameters of the transformation model 202 as predefined, static values. The values of the transformation model parameters can be selected in any appropriate way. For instance, the values of the transformation model parameters can be manually selected to define transformation functions that are expected to yield rich and informative transformed time series. As another example, the values of the transformation model parameters can be selected randomly, e.g., to implement random transformation functions, as described above.

An example of a technique for training the reconstruction model 200 is described in more detail below with reference to FIG. 5.

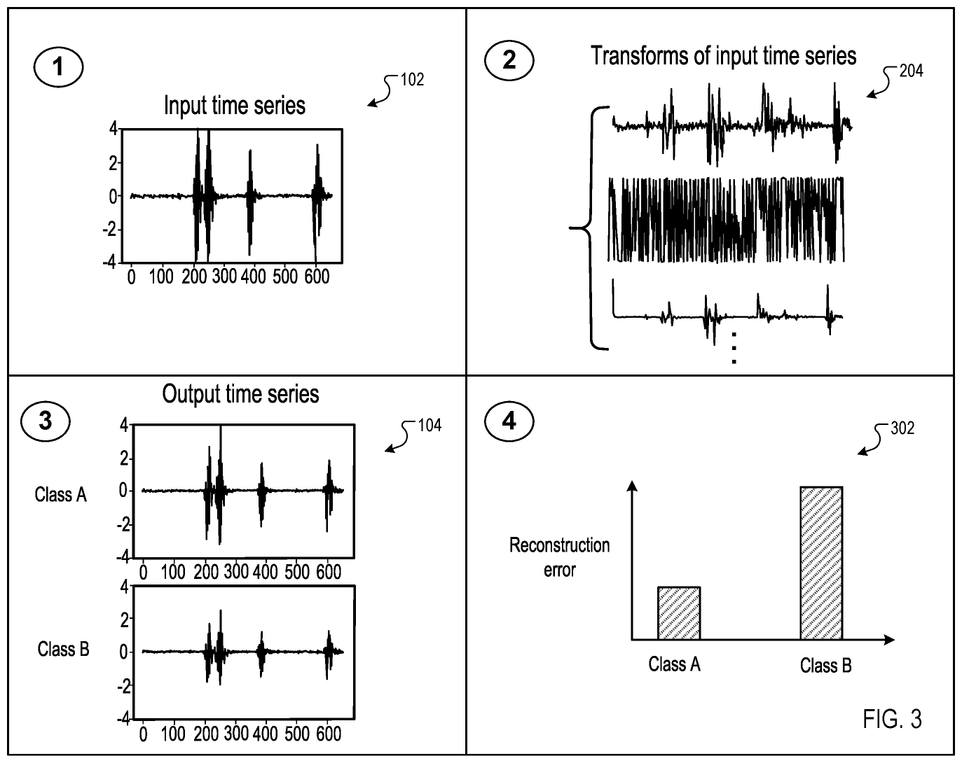

FIG. 3 provides an illustration of example operations that can be performed by the classification system 100.

At Step (1), shown at the top left of FIG. 3, the classification system 100 receives an input time series 102 to be classified into a set of classes. The input time series 102 can represent an audio waveform, radar data, a biomedical signal, video data, or any other appropriate time series.

At Step (2), shown at the top right of FIG. 3, the classification system 100 applies a set of transformation functions to the input time series 102 to generate a collection of transformed time series 204. For instance, if the input time series defines an audio waveform, then the classification system 100 can apply transformation functions such as a low-pass filter transformation function, a high-pass filter transformation function, an element-wise non-linear arctan transformation function, and the like. Generally, at least some of the transformation functions are non-linear functions. In some cases, the transformation functions are parametrized by static, untrained parameters that are chosen, e.g., randomly or based on domain knowledge, as opposed to being trained using machine learning techniques.

At Step (3), shown at the bottom left of FIG. 3, the classification system 100 processes the collection of transformed time series 204, using a projection model having a set of projection model parameters, to generate a respective output time series 104 corresponding to each class in the set of classes. For instance, the classification system 100 can generate each output time series 104 as a respective linear combination of the collection of transformed times series. The classification system 100 can train the projection model parameters to encourage the output time series 104 corresponding to the class of the input time series 102 to have a lower reconstruction error than the other output time series 104.

At Step (4), shown at the bottom right of FIG. 3, the classification system 100 determines a respective reconstruction error 302 for each output time series 104. The reconstruction error for an output time series 104 measures an error between: (i) the output time series 104, and (ii) the input time series 102. The classification system 100 can then classify the input time series 102 as being included in a class from the set of classes based on the reconstruction errors. For example, the classification system 100 can classify the input time series 102 as being included in the class corresponding to the output time series 104 having the lowest reconstruction error. For instance, the example illustrated in FIG. 3, the classification system 100 can classify the input time series 102 as being included in class A, i.e., because class A corresponds to the output time series 104 having the lowest reconstruction error.

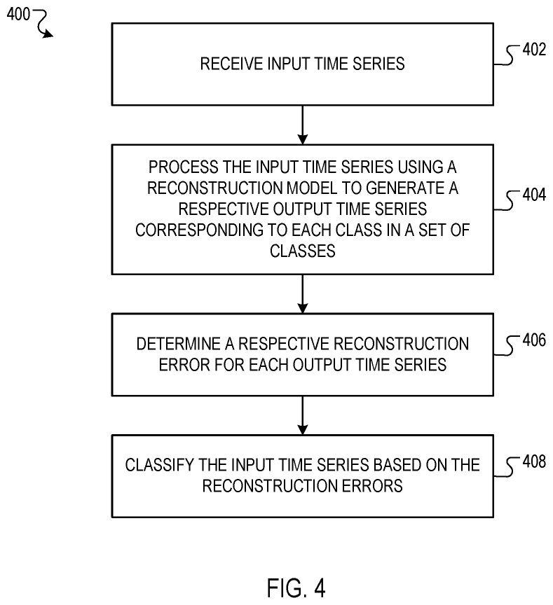

FIG. 4 is a flow diagram of an example process 400 for classifying an input time series into a class from a set of classes. For convenience, the process 400 will be described as being performed by a system of one or more computers located in one or more locations. For example, a classification system, e.g., the classification system 100 of FIG. 1, appropriately programmed in accordance with this specification, can perform the process 400.

The system receives the input time series (402). The input time series includes a respective sample at each time point in a sequence of time points.

The system processes the input time series using a reconstruction model to generate a reconstruction model output (404). The reconstruction model output includes a set of channels, where each channel corresponds to a respective class from the set of classes and defines a respective output time series that is a predicted reconstruction of the input time series.

The reconstruction model can include: (i) a transformation model, and (ii) a projection model.

The transformation model can include a set of transformation functions, where each transformation function operates on the time series to generate a corresponding transformed time series. The output of the transformation model can be represented as::

$$F ( X ) = ( \begin{matrix}{f}_{0} ( X ) \\ {f}_{1} ( X ) \\ ⋮ \\ {f}_{k} ( X )\end{matrix} ) \qquad ( 2 )$$

where X denotes the input time series, (fⱼ)ⱼ₌₁^{k} denote the transformation functions, and F(X) denotes the transformed time series stacked into a matrix.

The projection model can process the collection of transformed time series, i.e., generated by the transformation model, to generate the reconstruction model output. For example, the projection model can be defined by a matrix V that matrix multiplies the collection of transformed time series to generate the reconstruction model output. That is, the projection model can generate the reconstruction model output as:

R(X)=VF(X) (3)

where R(X) denotes the reconstruction model output for input time series X, V denotes a matrix defining the projection model, and F(X) denotes the collection of transformed time series generated by the training time series, e.g., as described with reference to equation (2). Generating the reconstruction model output by matrix multiplying the collection of transformed time series, e.g., as defined by equation (3), is equivalent to generating each channel of the reconstruction model output as a linear combination of the transformed time series.

The system determines a respective reconstruction error for each channel of the reconstruction model output based on an error between: (i) the output time series defined by the channel, and (ii) the input time series (406).

The system classifies the input time series as being included in a class from the set of classes based on the reconstruction errors (408). For example, the system can classify the input time series as being included in a class corresponding to a channel with a lowest reconstruction error from among the set of channels of the reconstruction model output.

FIG. 5 is a flow diagram of an example process training a reconstruction model. For convenience, the process 500 will be described as being performed by a system of one or more computers located in one or more locations. For example, a classification system, e.g., the classification system 100 of FIG. 1, appropriately programmed in accordance with this specification, can perform the process 500.

The system receive a set of training time series (502). Each training time series is associated with a respective class from the set of classes. The classification of each training time series can be determined, e.g., by manual human labeling, or in any other appropriate manner.

The system generates a respective target output for each training time series (504). The target output for a training time series defines a desired output to be generated by the reconstruction model by processing the training time series. The target output for a training time series can include a respective channel corresponding to each class in the set of classes. The channel corresponding to the class of the training time series can define the training time series itself, and each other channel (i.e., not corresponding to the class of the training time series) can define a default (e.g., predefined) time series. The default time series can be, e.g., a time series where each sample in the time series is a tensor of zeros (or some other default value).

The system determines transformation model parameters to optimize an objective function that, for each training time series, measures an error between: (i) the target output for the training time series, and (ii) the reconstruction model output generated by processing the training time series (506). For example, the objective function [symbol] can be given by:

$$ℒ = {\sum }_{i = 1}^{d} ‖ R ( {X}_{i} ) - {Y}_{i} ‖ + α ‖ {R}_{p} ‖ \qquad ( 4 )$$

where i indexes the training time series, d denotes the number of training time series, (Xᵢ)ᵢ₌₁^{d} denote the training time series, R(Xᵢ) denotes the reconstruction model output for training time series Xᵢ, Yᵢ denotes the target output for training time series Xᵢ, ∥⋅∥ denotes a norm, e.g., an L₂ norm, α is a scalar hyper-parameter, and ∥Rₚ∥ denotes a norm of some or all of the reconstruction model parameters. For instance, the reconstruction model parameters can be defined by a matrix V, as described with reference to equation (3), and ∥Rₚ∥ can be equal to an L₂ norm of the matrix V. The inclusion of a term in the loss function that measures a norm of the reconstruction model parameters can regularize and stabilize the training of the reconstruction model parameters.

The system can determine reconstruction model parameters to optimize the objective function in any of a variety of possible ways, i.e., using any appropriate machine learning training technique. For instance, for an objective function described by equation (4) and a reconstruction model described by equation (3), the system can determine an optimized matrix V* parameterizing the projection model as:

$${V}^{*} = {\hat{Y} ( \hat{R} - α I )}^{- 1} \qquad ( 5 )$$

$$\hat{Y} = {\sum }_{i = 1}^{d} {Y}_{i} {F ( {X}_{i} )}^{T} \qquad ( 6 )$$

$$\hat{R} = {\sum }_{i = 1}^{d} F ( {X}_{i} ) {F ( {X}_{i} )}^{T} \qquad ( 7 )$$

where i indexes the training time series, d denotes the number of training time series, (Xᵢ)iᵢ₌₁² denote the training time series, F(Xᵢ) denotes a matrix of transformed time series resulting from applying a set of transformation functions to training time series Xᵢ (as described with reference to equation (2)), and Yᵢ denotes the target output for training time series Xᵢ. Equations (5)-(7) provide an efficient one-step optimization for training the parameters of the projection model. In this example, the parameters of the transformation model are left static and untrained.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more computers for classifying an input time series into a class from a set of classes, the method comprising:
receiving an input time series comprising a respective sample at each time point in a sequence of time points;
processing the input time series using a reconstruction model to generate a reconstruction model output that comprises a plurality of channels,
wherein each channel of the plurality of channels defines a respective output time series that is a predicted reconstruction of the input time series, and
wherein each channel of the plurality of channels corresponds to a respective class from the set of classes;
determining a respective reconstruction error for each channel of the plurality of channels based on an error between: (i) the output time series defined by the channel, and (ii) the input time series; and
classifying the input time series as being included in a class from the set of classes based on the reconstruction errors.

2. The method of claim 1, wherein classifying the input time series as being included in a class from the set of classes based on the reconstruction errors comprises:
identifying a class corresponding to a channel with a lowest reconstruction error from among the plurality of channels; and
classifying the input time series as being included in the identified class.

3. The method of claim 1, wherein the reconstruction model comprises: (i) a transformation model including a set of transformation functions, and (ii) a projection model, and wherein processing the input time series using the reconstruction model to generate the reconstruction model output comprises:
processing the input time series using the transformation model to generate a collection of transformed time series, wherein each transformed time series results from applying a respective transformation function from the set of transformation functions to the input time series; and
processing the collection of transformed time series using the projection model to generate the reconstruction model output.

4. The method of claim 3, wherein the set of transformation functions comprises one or more non-linear transformation functions.

5. The method of claim 3, wherein the set of transformation functions comprises one or more of: a high-pass filter transformation function, a low-pass filter transformation function, a band-pass filter transformation function, a constant transformation function, an identity transformation function, or a lagging transformation function.

6. The method of claim 3, wherein processing the collection of transformed time series using the projection model to generate the reconstruction model output comprises:
generating each channel of the reconstruction model output as a respective linear combination of the collection of transformed time series.

7. The method of claim 3, wherein each transformed time series comprises a same number of samples as the input time series.

8. The method of claim 3, wherein the reconstruction model has been trained on a set of training time series, wherein the training encourages that, for each training time series, a channel of a reconstruction model output for the training time series that corresponds to a class of the training time series has a lower reconstruction error than each other channel of the reconstruction model output for the training time series.

9. The method of claim 8, wherein the training comprises, for each training time series:
generating a target output for the training time series, wherein the target output comprises a respective channel corresponding to each class from the set of classes, wherein:
the channel of the target output corresponding to a class of the training time series defines the training time series; and
each channel of the target output corresponding to a class different from the class of the training time series defines a default time series; and
training the reconstruction model to minimize an error between: (i) a reconstruction model output generated by processing the training time series using the reconstruction model, and (ii) the target output for the training time series.

10. The method of claim 9, wherein the default time series has a constant value of zero.

11. The method of claim 8, wherein the transformation model comprises a set of transformation model parameters, the projection model comprises a set of projection model parameters, and training the reconstruction model comprises:
training the projection model parameters while maintaining the transformation model parameters as static values.

12. The method of claim 1, wherein for each channel of the plurality of channels, the reconstruction error is based on an L₂ error between: (i) the output time series defined by the channel, and (ii) the input time series.

13. The method of claim 1, further comprising determining that the classification of the input time series satisfies a level of confidence defined by an error threshold.

14. The method of claim 13, wherein determining that the classification of the input time series satisfies the level of confidence defined by the error threshold comprises:
determining that a reconstruction error for the channel corresponding to the class into which the input time series has been classified is below the error threshold.

15. The method of claim 1, wherein the input time series represents an audio waveform.

16. The method of claim 1, wherein the input time series represents radar data.

17. The method of claim 1, wherein the input time series represents a biomedical signal.

18. The method of claim 17, wherein the biomedical signal comprises one or more of: a blood pressure signal, an electroencephalography (EEG) signal, an electrocardiogram (ECG) signal, or an electromyography (EMG) signal.

19. A system comprising:
one or more computers; and
one or more storage devices communicatively coupled to the one or more computers, wherein the one or more storage devices store instructions that, when executed by the one or more computers, cause the one or more computers to perform operations for classifying an input time series into a class from a set of classes, the operations comprising:
receiving an input time series comprising a respective sample at each time point in a sequence of time points;
processing the input time series using a reconstruction model to generate a reconstruction model output that comprises a plurality of channels,
wherein each channel of the plurality of channels defines a respective output time series that is a predicted reconstruction of the input time series, and
wherein each channel of the plurality of channels corresponds to a respective class from the set of classes;
determining a respective reconstruction error for each channel of the plurality of channels based on an error between: (i) the output time series defined by the channel, and (ii) the input time series; and
classifying the input time series as being included in a class from the set of classes based on the reconstruction errors.

20. One or more non-transitory computer storage media storing instructions that when executed by one or more computers cause the one or more computers to perform operations for classifying an input time series into a class from a set of classes, the operations comprising:
receiving an input time series comprising a respective sample at each time point in a sequence of time points;
processing the input time series using a reconstruction model to generate a reconstruction model output that comprises a plurality of channels,
wherein each channel of the plurality of channels defines a respective output time series that is a predicted reconstruction of the input time series, and
wherein each channel of the plurality of channels corresponds to a respective class from the set of classes;
determining a respective reconstruction error for each channel of the plurality of channels based on an error between: (i) the output time series defined by the channel, and (ii) the input time series; and
classifying the input time series as being included in a class from the set of classes based on the reconstruction errors.
